(19) United States
(12) Patent Application Publication
(10) Pub. No.: US 2011/0256068 A1
(43) Pub. Date: Oct. 20, 2011

(54) CONTRAST MEDIA COMPOSITIONS

(76) Inventors: Lars-Göran Wistand, Oslo (NO); Mikkel Thaning, Oslo (NO)

(21) Appl. No.: 13/142,309

(22) PCT Filed: Jan. 8, 2010
(86) PCT No.: PCT/EP10/50118

§ 371 Date: Jun. 27, 2011

(30) Foreign Application Priority Data
Jan. 9, 2009 (EP) 09150292.2

(51) Int. Cl. A61K 49/04; A61P 43/00

(52) U.S. Cl. 424/9.452

(57) ABSTRACT

Contrast media compositions comprising iodine containing contrast enhancing compounds of one or more monomeric non-ionic triiodinated aryl compounds and one or more dimeric non-ionic triiodinated aryl compounds. The bridge linking the two triiodinated aryl group of the dimeric compounds is substituted with a formyl function at least one nitrogen bridge atom.

### TECHNICAL FIELD OF THE INVENTION

The present invention relates to contrast media compositions where the contrast enhancing compounds are iodine containing compounds. More specifically the iodine containing compounds are chemical compounds containing single triiodinated phenyl groups and two linked triiodinated phenyl groups. The iodine containing compounds are non-ionic compounds which exist in molecular form in a carrier fluid.

The invention also relates to the use of such diagnostic compositions as contrast agents in diagnostic imaging and in particular in X-ray imaging, and to contrast media containing such compounds.

### DESCRIPTION OF RELATED ART

All diagnostic imaging is based on the achievement of different signal levels from different structures within the body. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast between a body structure and its surroundings the higher the quality of the images and the greater their value to the physician performing the diagnosis. Moreover, the greater the contrast the smaller the body structures that may be visualized in the imaging procedures, i.e. increased contrast can lead to increased spatial resolution.

The diagnostic quality of images is strongly dependent on the inherent noise level in the imaging procedure, and the ratio of the contrast level to the noise level can thus be seen to represent an effective diagnostic quality factor for diagnostic images.

Achieving improvement in such a diagnostic quality factor has long been and still remains an important goal. In techniques such as X-ray, magnetic resonance imaging (MRI) and ultrasound, one approach to improving the diagnostic quality factor has been to introduce contrast enhancing materials formulated as contrast media into the body region being imaged.

Thus, early examples of X-ray contrast agents were insoluble inorganic barium salts which enhanced X-ray attenuation in the body zones into which they distributed. For the last 50 years the field of X-ray contrast agents has been dominated by soluble iodine containing compounds. Commercial available contrast media containing iodinated contrast agents are usually classified as ionic monomers such as diatrizoate (marketed e.g. under the trade mark Gastrografen™), ionic dimers such as ioxaglate (marketed e.g. under the trade mark Hexabrix™), nonionic monomers such as iohexol (marketed e.g. under the trade mark Omnipaque™), iopamidol (marketed e.g. under the trade mark Isovue™), iomeprol (marketed e.g. under the trade mark Iomeron™) and the non-ionic dimer iodixanol (marketed under the trade mark Visipaque™).

The most widely used commercial non-ionic X-ray contrast agents such as those mentioned above are considered safe. Contrast media containing iodinated contrast agents are used in more than 20 millions of X-ray examinations annually in the USA and the number of adverse reactions is considered acceptable. However, since a contrast enhanced X-ray examination will require up to about 200 ml contrast media administered in a total dose, there is a continuous drive to provide improved contrast media.

The utility of the contrast media is governed largely by its toxicity, by its diagnostic efficacy, by adverse effects it may have on the subject to which the contrast medium is administered, and by the ease of production, storage and administration. Since such media are conventionally used for diagnostic purposes rather than to achieve direct therapeutic effect, it is generally desirable to provide media having as little as possible effect on the various biological mechanisms of the cells or the body as this will lead to lower toxicity and lower adverse clinical effect. The toxicity and adverse biological effects of a contrast medium are contributed to by the components of the formulation medium, e.g. the solvent or carrier as well as the contrast agent itself and its components such as ions for the ionic contrast agents and also by its metabolites.

The major contributing factors to the toxicity of the contrast medium are identified as the chemotoxicity of the contrast agent, the osmolality of the contrast medium and the ionic composition or lack thereof of the contrast medium. Desirable characteristics of an iodinated contrast agent are low toxicity of the compound itself (chemotoxicity), low viscosity of the contrast medium wherein the compound is dissolved, low osmolality of the contrast medium and a high iodine content (frequently measured in mg iodine per ml of the formulated contrast medium for administration). The iodinated contrast agent must also be completely soluble in the formulation medium, usually an aqueous medium, and remain in solution during storage.

The osmolalities of the commercial products, and in particular of the non-ionic compounds, is acceptable for most media containing dimers and non-ionic monomers although there is still room for improvement. In coronary angiography for example, injection into the circulatory system of a bolus dose of contrast medium has caused severe side effects. In this procedure contrast medium rather than blood flows through the system for a short period of time, and differences in the chemical and physiochemical nature of the contrast medium and the blood that it replaces can cause undesirable adverse effects such as arrhythmias, QT prolongation and reduction in cardiac contractive force. Such effects are seen in particular with ionic contrast agents where osmotoxic effects are associated with hypertonicity of the injected contrast medium. Contrast media that are isotonic or slightly hypotonic with the body fluids are particularly desired. Low osmolar contrast media have low renal toxicity which is particularly desirable. The osmolality is a function of the number of particles per volume unit of the formulated contrast medium. The part of the patient population considered as high risk patients is increasing. To meet the need for continuous improvement of in vivo X-ray diagnostic agents for the entire patient population, there is a continuous drive in finding X-ray contrast compositions that have improved properties, also with regards to contrast induced nephrotoxicity (CIN).

To keep the injection volume of the contrast media as low as possible it is highly desirable to formulate contrast media with high concentration of iodine/ml, and still maintain the osmolality of the media at a low level, preferably below or close to isotonicity. The development of non-ionic monomeric contrast agents and in particular non-ionic bis(triiodophenyl) dimers such as iodixanol (EP patent 108638) has provided contrast media with reduced osmotoxicity allowing contrast effective iodine concentration to be achieved with hypotonic solution, and has even allowed correction of ionic imbalance by inclusion of plasma ions while still maintaining the contrast medium Visipaque™ at the desired osmolality (WO 90/01194 and WO 91/13636).

Commercially available X-ray contrast media marketed with high iodine concentration have relative high viscosity, ranging from about 15 to about 60 mPas at ambient temperature. Generally, at equal iodine concentration, contrast media for which the contrast enhancing agent is a dimer has higher viscosity than the corresponding contrast media wherein the contrast enhancing agent is a monomer corresponding to the dimer. Such high viscosities may pose problems to the administrators of the contrast medium, requiring relatively large bore needles or high applied pressure, and such problems are particularly pronounced in pediatric radiography and in radiographic techniques which require rapid bolus administration, e.g. in angiography.

WO94/14478 (Dibra S.p.A/Bracco S.p.A.) suggests injectable aqueous solutions of mixtures of non-ionic and water-soluble iodinated aromatic compounds comprising an aromatic nucleus which is at least triiodo substituted and compounds comprising at least two aromatic nuclei variably bound together, each one at least triiodo substituted.

WO2005/087272 (Mallinckrodt Inc) proposes mixtures of iodinated contrast agents, in particular comprising the dimeric iodinated contrast agent iosmin.

No X-ray contrast media compositions comprising both a non-ionic dimeric contrast agent compound and a non-ionic monomeric contrast agent compound are on the market.

Hence there still exists a desire to develop contrast media compositions having a high concentration of iodine and at the same time having improved properties over the soluble iodine containing compounds on the market in one or more of the following properties: renal toxicity, osmolality, viscosity, solubility, injection volumes/iodine concentration and attenuation/radiation dose and any additional adverse effect known or discovered for such iodinated compounds. The compositions should be stable under storage in dry form and/or in solution, and ease and economy in manufacture is an additional desired property. In particular there is a desire to develop contrast media compositions having a high concentration of iodine per volume unit and still maintaining a manageable viscosity and an acceptable osmolarity.

### SUMMARY OF THE INVENTION

The present invention provides contrast media compositions having improved properties over the known media with regards to at least one of the criteria mentioned above and in particular to osmolality and viscosity and specifically to viscosity. The contrast media compositions comprise iodine containing contrast enhancing compounds where iodine containing compounds are chemical compounds containing single triiodinated phenyl entities of formula (I) and two linked triiodinated phenyl entities of formula (II) as defined hereinafter. The compounds of formulas (I) and (II) are non-ionic contrast agents which exist in molecular form in a carrier fluid. By providing contrast media compositions comprising non-ionic iodinated monomeric compounds and non-ionic iodinated dimeric compounds, it is possible to provide contrast agent compositions containing an iodine concentration of more than 320 mgl/ml of the X-ray contrast media composition in ready to use form and still maintaining the osmolarity and viscosity at acceptable levels. It has been found that mixtures of monomeric and dimeric compounds of formula (I) and (II) have lower viscosities than would be expected for each of the solutions of the monomeric and dimeric compounds at the same concentrations in mgl/ml.

### DETAILED DESCRIPTION OF THE INVENTION

The new compositions of the invention, their use as X-ray contrast agents, their formulation and production are specified in the attached claims and in the specification hereinafter. The compositions of the invention comprise mixtures of both monomeric compounds and dimeric compounds.

The contrast enhancing compounds of the single triiodinated phenyl groups, denoted monomeric compounds, comprises compounds of the general formula (I)

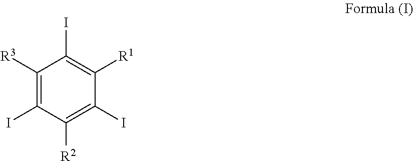

and salts or optical active isomers thereof,

wherein each of R¹, R² and R³ are the same or different and denotes a hydrogen atom or a non-ionic hydrophilic moiety, provided that at least one of the R¹, R² and R³ groups in the compound of formula (I) is a hydrophilic moiety.

The contrast enhancing compounds of two linked triiodinated phenyl groups, denoted non-ionic dimeric compounds, are synthetic chemical compounds of formula (II)

R—N(CHO)—X—N(R⁶)—R Formula (II)

and salts or optical active isomers thereof,
wherein

X denotes a C₃ to C₈ straight or branched alkylene moiety optionally with one or two CH₂ moieties replaced by oxygen atoms, sulphur atoms or NR⁴ groups and wherein the alkylene moiety optionally is substituted by up to six —OR⁴ groups;

R⁴ denotes a hydrogen atom or a C₁ to C₄ straight or branched alkyl group;

R⁶ denotes a hydrogen atom or an acyl function; and

each R independently is the same or different and denotes a triiodinated phenyl group, preferably a 2,4,6-triiodinated phenyl group, further substituted by two groups R⁵ wherein each R⁵ is the same or different and denotes a hydrogen atom or a non-ionic hydrophilic moiety, provided that at least one R⁵ group in the compound of formula (II) is a hydrophilic moiety.

In formula (I) above, the non-ionic hydrophilic moieties R¹, R² and R³ may be any of the non-ionizing groups conventionally used to enhance water solubility. Hence, the R¹, R² and R³ substituents may be the same or different and shall preferably all denote a non-ionic hydrophilic moiety comprising esters, amides and amine moieties, optionally further substituted by a straight chain or branched chain C₁₋₁₀ alkyl groups, preferably C₁₋₅ alkyl groups, where the alkyl groups also may have one or more CH₂ or CH moieties replaced by oxygen or nitrogen atoms. The R¹, R² and R³ substituents may also further contain one or more groups selected from oxo, hydroxyl, amino or carboxyl derivative, and oxo substituted sulphur and phosphorus atoms. Each of the straight or branched alkyl groups preferably contains 1 to 6 hydroxy groups and more preferably 1 to 3 hydroxy groups. Therefore, in a further preferred aspect, the R¹, R² and R³ substituents are the same or different and are polyhydroxy C₁₋₅ alkyl, hydroxyalkoxyalkyl with 1 to 5 carbon atoms and hydroxypolyalkoxyalkyl with 1 to 5 carbon atoms, and are attached to the iodinated phenyl group via amide and carbamoyl linkages.

The R¹, R² and R³ groups of the formulas listed below are particularly preferred:

—CON H₂

—CONHCH₃

—CONH—CH₂—CH₂—OH

—CONH—CH₂—CH₂—OCH₃

—CONH—CH₂—CHOH—CH₂—OH

—CONH—CH₂—CHOCH₃—CH₂—OH

—CONH—CH₂—CHOH—CH₂—OCH₃

—CON(CH₃)CH₂—CHOH—CH₂OH

—CONH—CH—(CH₂—OH)₂

—CON—(CH₂—CH₂—OH)₂

—CON—(CH₂—CHOH—CH₂—OH)₂

—CONH—OCH₃

—CON(CH₂—CHOH—CH₂—OH)(CH₂—CH₂—OH)

—CONH—C(CH₂—OH)₂CH₃,

—CONH—C(CH₂—OH)₃, and

—CONH—CH(CH₂—OH)(CHOH—CH₂—OH)

—NH(COCH₃)

—N(COCH₃)C₁₋₃ alkyl

—N(COCH₃)-mono, bis or tris-hydroxy C₁₋₄ alkyl

—N(COCH₂OH)-hydrogen, C₁₋₄ alkyl, mono, bis or tris-hydroxy C₁₋₄ alkyl

—N(CO—CHOH—CH₂OH)-hydrogen, mono, bis or trihydroxylated C₁₋₄ alkyl

—N(CO—CHOH—CHOH—CH₂OH)-hydrogen, mono, bis or trihydroxylated C₁₋₄ alkyl

—N(CO—CH—(CH₂OH)₂)-hydrogen, mono, bis or trihydroxylated C₁₋₄ alkyl

—N(CO—CHOH—CH₃)-hydrogen, mono, bis or trihydroxylated C₁₋₄ alkyl

—NH(CO—CH₂OCH₃) and

—N(COCH₂OH)₂

Even more preferred, two of the R¹, R² and R³ groups are equal and denote one or more moieties of the formulas

—CONH—CH₂—CH₂—OH,

—CONH—CH₂—CHOH—CH₂—OH,

—CON(CH₃)CH₂—CHOH—CH₂OH,

—CONH—CH—(CH₂—OH)₂ and

—CON—(CH₂—CH₂—OH)₂, while the third group of R¹, R² and R³ denotes

—N(COCH₃)-mono, bis or tris-hydroxy C₁₋₄ alkyl,

—N(COCH₂OH)-hydrogen, C₁₋₄ alkyl , mono, bis or tris-hydroxy C₁₋₄ alkyl,

—N(CO—CHOH-CH₃)-hydrogen, mono, bis or trihydroxylated C₁₋₄ alkyl, or

—N(CO—CH—(CH₂OH)₂)-hydrogen, mono, bis or trihydroxylated C₁₋₄ alkyl;

Particularly preferred are the monomeric compounds described in WO97/00240 and in particular the compound BP257 of example 2, and additionally the commercially available compounds iopamidol, iomeprol, ioversol, iopromide, ioversol, iobitridol, iopentol and iohexol. Most particularly preferred are the compounds iopamidol and iohexol.

Compounds mentioned above can be produced following synthesis known from the literature, see e.g. U.S. Pat. Nos. 4,352,788, 4,364,921, 4,001,323, 4,341,756, 4,250,113, 5,035,877, 5,043,152 and the patent application WO97/00240.

In formula (II) above, X preferably denotes a straight C₃ to C₈ alkylene chain optionally substituted by one to six —OR⁴ groups. More preferably X denotes a straight C₃ to C₅ alkylene chain having at least one —OR⁴ group, preferably at least one hydroxyl group in a position that is not vicinal to the bridge nitrogen atom. More preferably the alkylene chain is substituted by one to three hydroxyl groups and still more preferably the alkylene chain is a straight propylene, butylene or pentylene chain substituted by one, two or three hydroxyl groups. Particularly preferred groups X are selected from 2-hydroxy propylene, 2,3-dihydroxy butylene, 2,4-dihydroxy pentylene and 2,3,4-trihydroxy pentylene, and most particularly X is the 2-hydroxy propylene entity.

R⁴ preferably denotes a hydrogen atom or a methyl group, most preferably a hydrogen atom.

The substituent R⁶ preferably denotes a hydrogen atom or a residue of an aliphatic organic acid, and in particular a C₁ to C₅ organic acid such as formyl, acetyl, propionyl, butyryl, isobutyryl and valeriyl moieties. Hydroxylated and metoxylated acyl moieties are also feasible. In a particularly preferred embodiment the R⁶ group in the compound of formula (II) denotes a hydrogen atom, the formyl moiety or the acetyl moiety, most preferably the formyl moiety.

Each of the iodinated R groups can be the same or different and preferably denote a 2,4,6-triiodinated phenyl group, further substituted by two groups R⁵ in the remaining 3 and 5 positions in the phenyl moiety.

The non-ionic hydrophilic moieties , R⁵, may be any of the non-ionizing groups conventionally used to enhance water solubility. Hence, the R⁵ substituents may be the same or different and shall preferably all denote a non-ionic hydrophilic moiety comprising esters, amides and amine moieties, optionally further substituted by a straight chain or branched chain C₁₋₁₀ alkyl groups, preferably C₁₋₅ alkyl groups, where the alkyl groups also may have one or more CH₂ or CH moieties replaced by oxygen or nitrogen atoms. The R⁵ substituents may also further contain one or more groups selected from oxo, hydroxyl, amino or carboxyl derivative, and oxo substituted sulphur and phosphorus atoms. Each of the straight or branched alkyl groups preferably contains 1 to 6 hydroxy groups and more preferably 1 to 3 hydroxy groups. Therefore, in a further preferred aspect, the R⁵ substituents are the same or different and are polyhydroxy C₁₋₅ alkyl, hydroxyalkoxyalkyl with 1 to 5 carbon atoms and hydroxypolyalkoxyalkyl with 1 to 5 carbon atoms, and are attached to the iodinated phenyl group via an amide or a carbamoyl linkage, preferably amide linkages.

The R⁵ groups of the formulas listed below are particularly preferred:

—CONH₂

—CONHCH₃

—CONH—CH₂—CH₂—OH

—CONH—CH₂—CH₂—OCH₃

—CONH—CH₂—CHOH-CH₂—OH

—CONH—CH₂—CHOCH₃—CH₂—OH

—CONH—CH₂—CHOH—CH₂—OCH₃

—CON(CH₃)CH₂—CHOH—CH₂OH

—CONH—CH—(CH₂—OH)₂

—CON—(CH₂—CH₂—OH)₂

—CON—(CH₂—CHOH—CH₂—OH)₂

—CONH—OCH₃

—CON(CH₂—CHOH—CH₂—OH)(CH₂—CH₂—OH)

—CONH—C(CH₂—OH)₂CH₃,

—CONH—C(CH₂—OH)₃, and

—CONH—CH(CH₂—OH)(CHOH—CH₂—OH)

—NH(COCH₃)

—N(COCH₃)C₁₋₃ alkyl

—N(COCH₃)-mono, bis or tris-hydroxy C₁₋₄ alkyl

—N(COCH₂OH)-hydrogen, mono, bis or tris-hydroxy C₁₋₄ alkyl

—N(CO—CHOH—CH₂OH)-hydrogen, mono, bis or trihydroxylated C₁₋₄ alkyl.

—N(CO—CHOH—CHOH—CH₂OH)-hydrogen, mono, bis or trihydroxylated C₁₋₄ alkyl

—N(CO—CH—(CH₂OH)₂)-hydrogen, mono, bis or trihydroxylated C₁₋₄ alkyl; and

—N(COCH₂OH)₂

Even more preferably the R⁵ groups will be equal or different and denote one or more moieties of the formulas —CONH—CH₂—CH₂—OH, —CONH—CH₂—CHOH—CH₂—OH, —CON(CH₃)CH₂—CHOH—CH₂OH, —CONH—CH—(CH₂—OH)₂ and —CON—(CH₂—CH₂—OH)₂. Still more preferably both R groups are the same and the R² groups in each R are the same or different and denote —CONH—CH₂—CH₂—OH, —CONH—CH₂—CHOH—CH₂—OH, CON(CH₃)CH₂—CHOH—CH₂OH, —CON—(CH₂—CH₂—OH)₂ and —CONH—CH—(CH₂—OH)₂ In a particularly preferred embodiment, both R groups are the same and all R⁵ groups denote the entity of formula —CONH—CH₂—CHOH—CH₂—OH.

Thus, preferred non-ionic dimeric compounds of the compositions according to the invention include the compounds of formula (IIa-c):

Formula (IIa-c)

R—N(CHO)—X—N(CHO)—R (IIa)

R—N(CHO)—X—N(CO(CH₃))—R (IIb)

R—N(CHO)—X—NH—R (IIc)

In formula (IIa-c), each group R has the meaning above, more preferably both iodophenyl groups R are the same and the R⁵ groups all denote non-ionic hydrophilic moieties, and preferably the R⁵ groups are linked to iodinated phenyl moiety by amide linkages. X preferably denotes straight chain alkylene groups with 3 to 5 carbon atoms and having one to three hydroxyl substituents at positions that are not adjacent to the nitrogen function.

Compounds of formula (IIa) are particularly preferred, in particular compounds having a monohydroxylated alkylene bridge X, in particularly a monohydroxylated propylene bridge. Some preferred examples according to the invention include the compounds of formulas (III a) to (III u) below.

5,5′-(2-hydroxypropane-1,3-diyl)bis(formylazanediyl)bis(N¹,N³-bis(2,3-dihydroxypropyl)-2,4,6-triiodoisophthalamide):

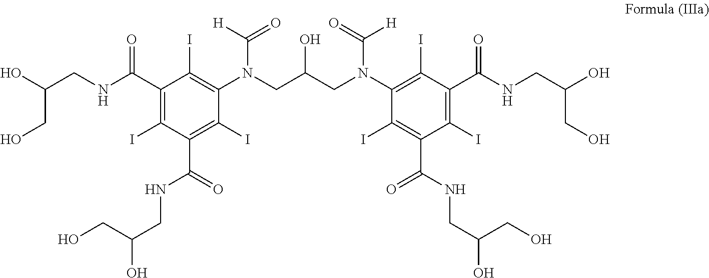

5,5′-(2,3-dihydroxybutane-1,4-diyObis(formylazanediyObis(N¹,N³-bis(2,3-dihydroxypropyl)-2,4,6-triiodoisophthalamide):

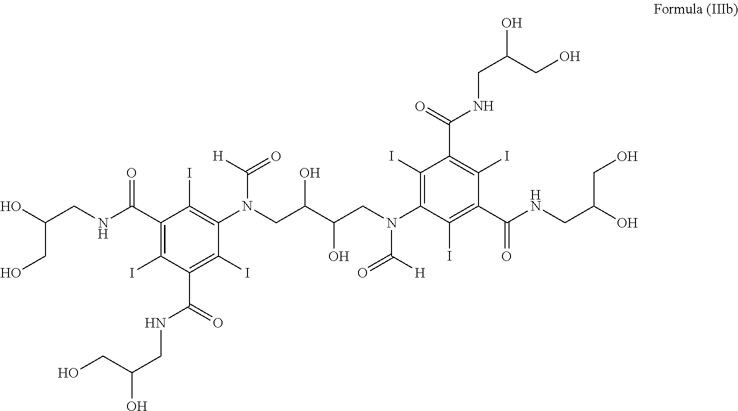

5,5′-(2,4-dihydroxypentane-1,5-diyl)bis(formylazanediyl)bis(N¹,N³-bis(2,3-dihydroxypropyl)-2,4,6-triiodoisophthalamide):

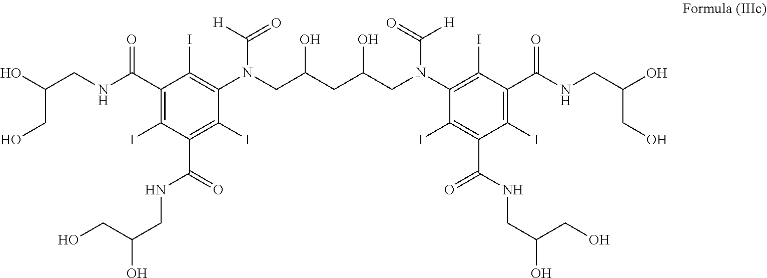

5,5′-(2-hydroxypropane-1,3-diyl)bis(formylazanediyl)bis(N¹,N³-bis(1,3-dihydroxypropan-2-yl)-2,4,6-triiodoisophthalamide):

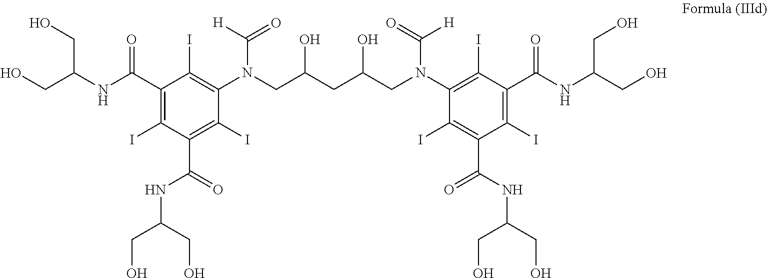

5,5′-(2,3-dihydroxybutane-1,4-diyl)bis(formylazanediyl)bis(N¹,N³-bis(1,3-dihydroxypropan-2-yl)-2,4,6-triiodoisophthalamide):

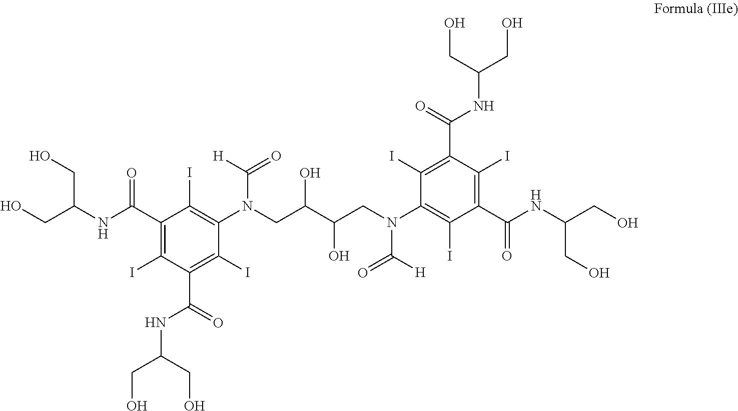

5,5′-(2,4-dihydroxypentane-1,5-diyl)bis(formylazanediyl)bis(N¹,N³-bis(1,3-dihydroxypropan-2-yl)-2,4,6-triiodoisophthalamide):

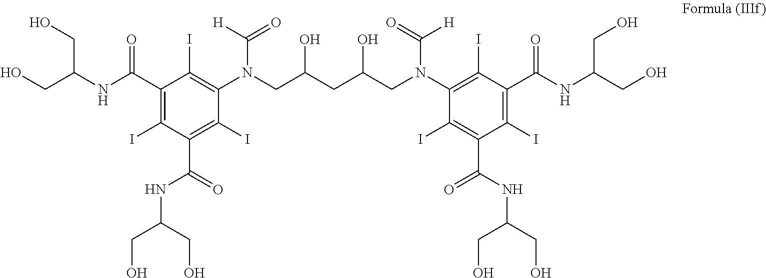

5,5′-(2,3,4-trihydroxypentane-1,5-diyl)bis(formylazanediyl)bis(N¹,N³-bis(2,3-dihydroxypropyl)-2,4,6-triiodoisophthalamide):

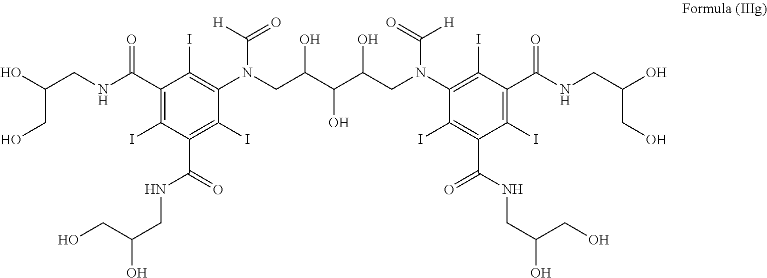

5,5′-(2,3-dihydroxybutane-1,4-diyl)bis(formylazanediyl)bis(N¹,N³-bis(2,3-dihydroxypropyl)-2,4,6-thiodo-N¹,N³-dimethylisophthalamide):

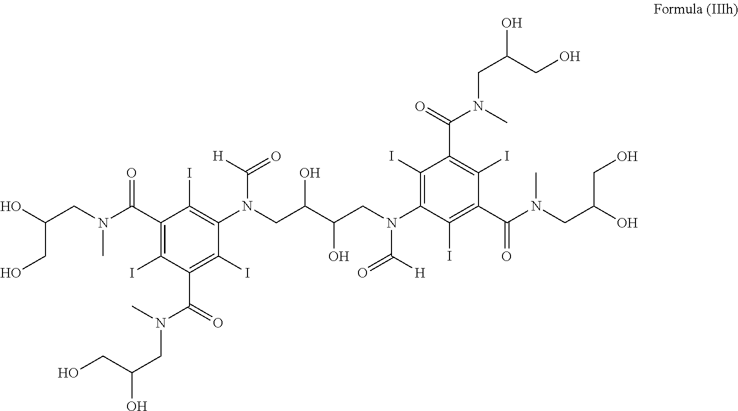

5,5′-(2,4-dihydroxypentane-1,5-diyl)bis(formylazanediyl)bis(N¹,N³-bis(2,3-dihydroxypropyl)-2,4,6-thiodo-N¹,N³-dimethylisophthalamide):

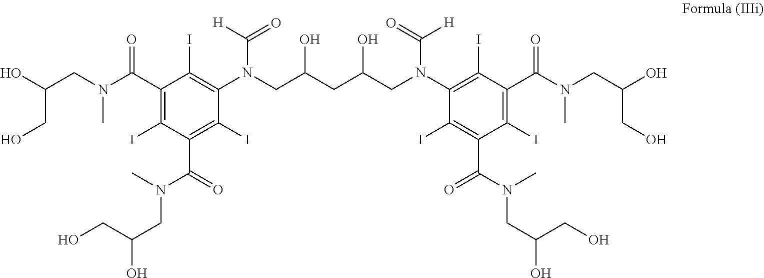

5,5′-(2-hydroxypropane-1,3-diyl)bis(formylazanediyl)bis(N¹,N³-bis(2,3-dihydroxypropyl)-2,4,6-thiodo-N¹,N³-dimethylisophthalamide):

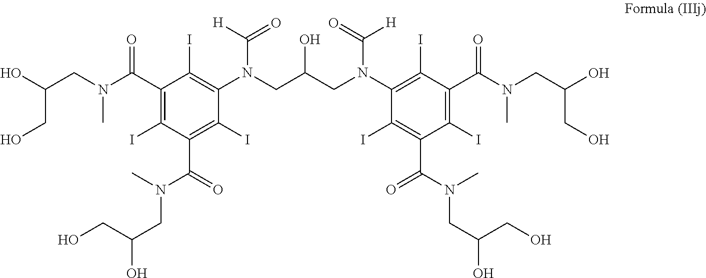

5,5′-(2,3-dihydroxybutane-1,4-diyl)bis(formylazanediyl)bis(N¹-(2,3-dihydroxypropyl)-N³-(2-hydroxyethyl)-2,4,6-triiodoisophthalamide):

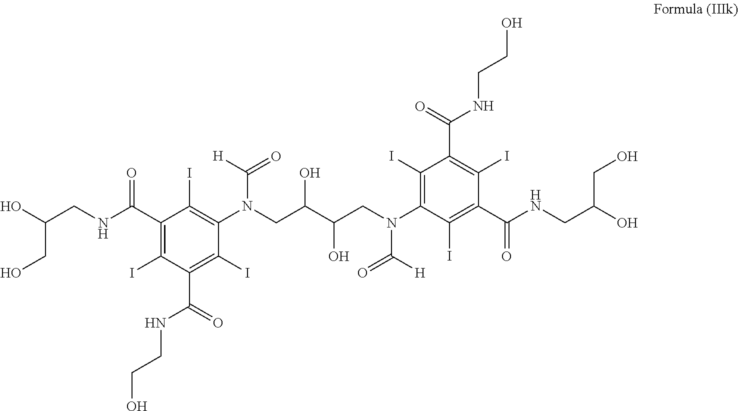

5,5′-(2-hydroxypropane-1,3-diyl)bis(formylazanediyl)bis(N¹-(2,3-dihydroxypropyl)-N³-(2-hydroxyethyl)-2,4,6-triiodoisophthalamide):

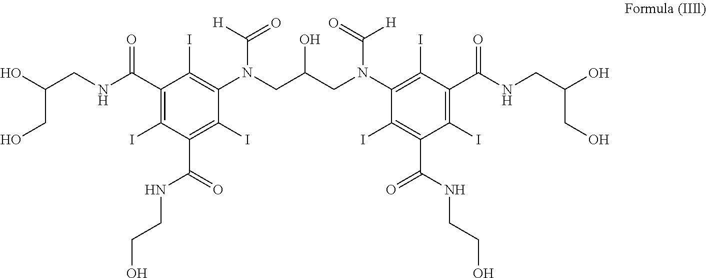

5,5′-(2,4-dihydroxypentane-1,5-diyl)bis(formylazanediyl)bis(N¹-(2,3-dihydroxypropyl)-N³-(2-hydroxyethyl)-2,4,6-triiodoisophthalamide):

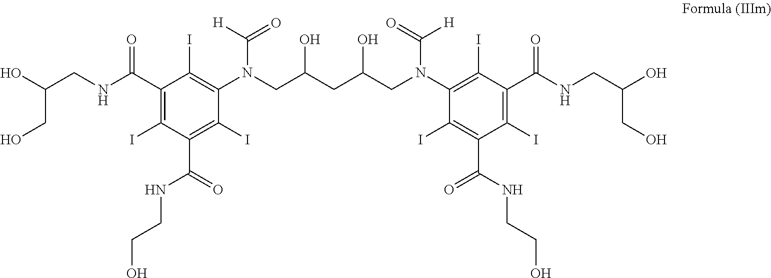

5,5′-(2-hydroxypropane-1,3-diyl)bis(formylazanediyl)bis(N¹-(1,3-dihydroxypropan-2-yl)-N³-(2,3-dihydroxypropyl)-2,4,6-triiodoisophthalamide):

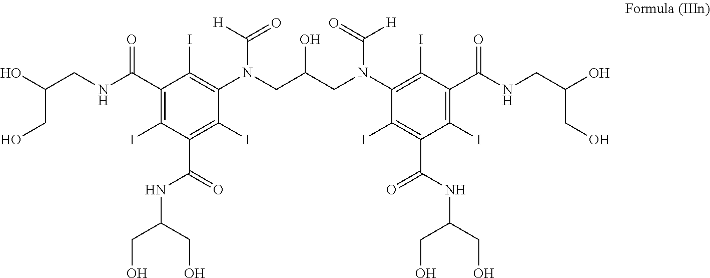

5,5′-(2,3-dihydroxybutane-1,4-diyl)bis(formylazanediyl)bis(N¹-(1,3-dihydroxypropan-2-yl)-N³-(2,3-dihydroxypropyl)-2,4,6-triiodoisophthalamide):

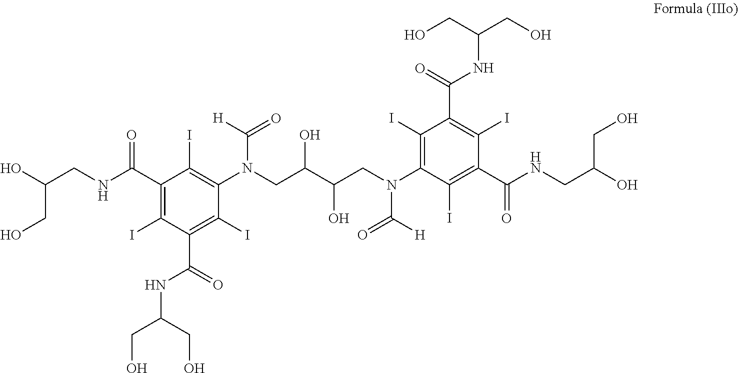

5,5′-(2,4-dihydroxypentane-1,5-diyl)bis(formylazanediyl)bis(N¹-(1,3-dihydroxypropan-2-yl)-N³-(2,3-dihydroxypropyl)-2,4,6-triiodoisophthalamide):

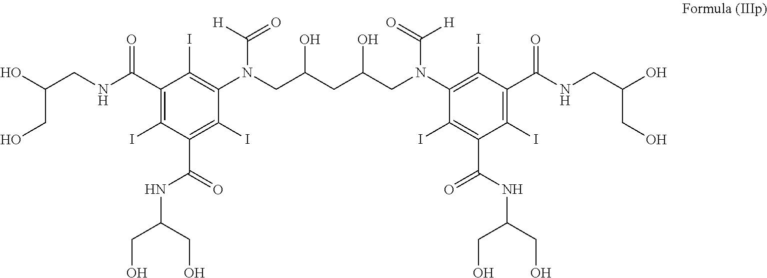

5,5′-(2-hydroxypropane-1,3-diyl)bis(formylazanediyl)bis(N¹-(1,3-dihydroxypropan-2-yl)-N³-(2-hydroxyethyl)-2,4,6-triiodoisophthalamide):

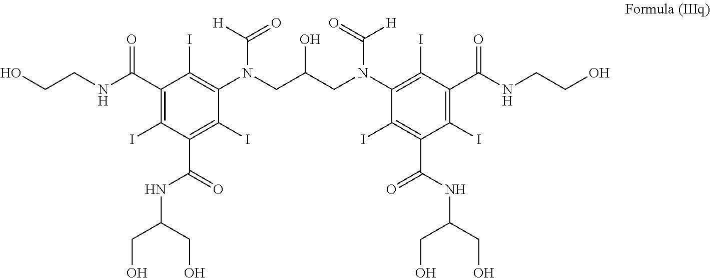

5,5′-(2,3-dihydroxybutane-1,4-diyl)bis(formylazanediyl)bis(N¹-(1,3-dihydroxypropan-2-yl)-N³-(2-hydroxyethyl)-2,4,6-triiodoisophthalamide):

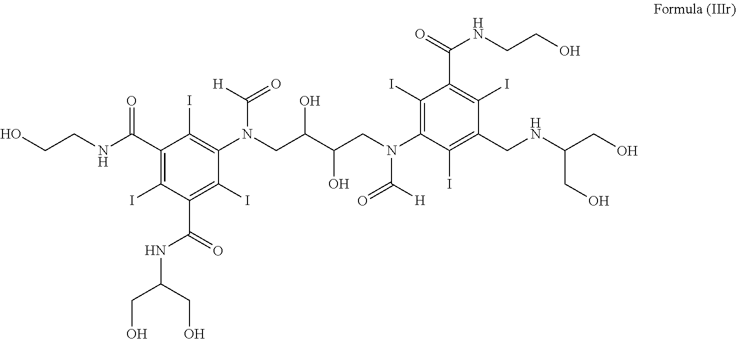

5,5′-(2,4-dihydroxypentane-1,5-diyl)bis(formylazanediyl)bis(N¹-(1,3-dihydroxypropan-2-yl)-N³-(2-hydroxyethyl)-2,4,6-triiodoisophthalamide):

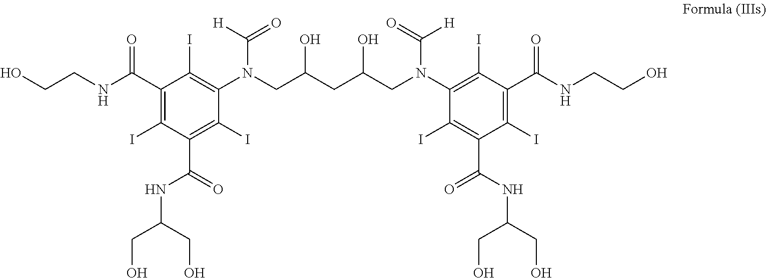

5-(N-(3-(N-(3,5-bis(2,3-dihydroxypropylcarbamoyl)-2,4,6-triiodophenyl)acetamido)-2-hydroxypropyl)formamido)-N¹,N³-bis(2,3-dihydroxypropyl)-2,4,6-triiodoisophthalamide:

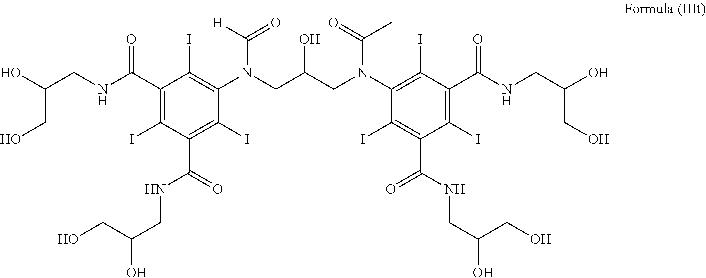

5-(3-(N-(3,5-bis(2,3-dihydroxypropylcarbamoyl)-2,4,6-triiodophenyl)formamido)-2-hydroxypropylamino)-N¹,N³-bis(2,3-dihydroxypropyl)-2,4,6-triiodoisophthalamide:

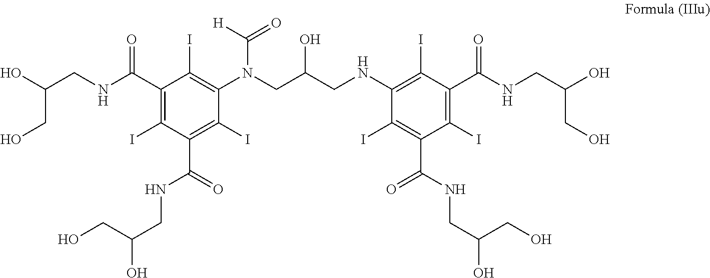

Most preferred is the compound of formula IIIa above.

Hence, more particularly, the invention provides contrast media compositions comprising a mixture of iodine containing contrast enhancing compounds wherein one or more compounds are of formula (I) (denoted monomeric compounds)

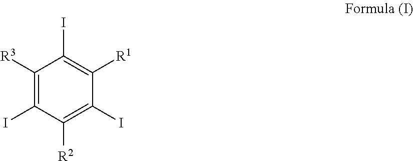

and salts or optical active isomers thereof,
wherein each of R¹, R² and R³ have the meanings denoted above,
and one or more of the compounds are of formula (II) (denoted dimeric compounds)

Formula (II)

R—N(CHO)—X—N(R⁶)—R (II)

and salts or optical active isomers thereof,
wherein each of X, R and R⁶ have the meanings denoted above.

Such compositions provide useful contrast media compositions.

In particular, compositions wherein the monomeric compounds of formula (I) are selected from one or more of the compounds BP257, iopamidol, iomeprol, ioversol, iopromide, iobitridol, iopentol and iohexol, more preferably from the compounds iopamidol and iohexol, and the dimeric compounds are selected from one or more of those of formula III(a) to III(u) above, more preferably the compound of formula III(a), provide useful contrast media compositions.

The compositions should include the monomeric compound of formula (I) in an amount of from at least 1 weight % and up to 40 weight % of the total iodine content. In the same way, the compositions should include the dimeric compound of formula (II) in an amount of from at least 60 weight % and up to 99 weight % of the total iodine content.

More preferably, the amount of monomeric compounds in the composition is from 5 to 35 weight %, even more preferably from 10 to 30 weight % and still more preferably about 20 weight % of the total iodine content.

More preferably, the amount of dimeric compounds in the composition is from 95 to 65 weight %, even more preferably from 90 to 70 weight % and still more preferably about 80 weight % of the total iodine content.

The preferred amounts of monomeric and dimeric compounds are decided based on an optimization of the amounts that provide the optimal viscosity of the composition and/or the optimal osmolarity of the composition and/or the optimal iodine content of the composition, preferably the relative amounts of monomeric and dimeric compounds are decided based on the preferred combinations providing a lowest possible viscosity at an amount of about 350 mgl/ml and having acceptable osmolarities, e.g. being isoosmolar or hypoosmolar if necessary to allow for the addition of salts as explained below.

Generally for compositions in a ready to use form the iodine content of the compositions should preferably be at least 320 mgl/ml, more preferably at least 335 mgl/ml and even more preferable at least 350 mgl/ml.

For contrast media compositions which are administered by injection or infusion, the desired upper limit for the solution's viscosity at ambient temperature (20° C.) is about 40 mPas, however viscosities of up to 50 to 60 mPas and even more than 60 mPas can be tolerated.

The compositions should preferably have a viscosity of below 40mPas at 20° C., more preferably below 30mPas at 20° C., for example between 25 and 30 mPas at 20° C.

For contrast media given by bolus injection, e.g. in angiographic procedures, osmotoxic effects must be considered and preferably the osmolality should be below 1 Osm/kg H₂O, preferably below 850 mOsm/kg H₂O and more preferably below 500 mOsm/kg H₂O, and even more preferably about 300 mOsm/kg H₂O.

With the compositions of the invention such viscosity, osmolality and iodine concentrations targets can be met. Indeed, effective iodine concentrations can be reached with hypotonic solutions. It may thus be desirable to make up the solution's tonicity by the addition of plasma cations so as to reduce the toxicity contribution that derives from the imbalance effects following bolus injection. Such cations will desirably be included in the ranges suggested in WO 90/01194 and WO 91/13636. In particular, addition of sodium and calcium ions to provide a contrast medium isotonic with blood for all iodine concentrations is desirable and obtainable. The plasma cations may be provided in the form of salts with physiologically tolerable counterions, e.g. chloride, sulphate, phosphate, hydrogen carbonate etc., with plasma anions preferably being used.

The compositions of the invention are compositions for diagnostic use, in particular for X-ray diagnostic use. The composition comprises at least one compound of formula (I) and at least one compound of formula (II) as described above and will usually be formulated with at least one physiologically tolerable carrier or excipient, e.g. in aqueous solution for injection optionally together with added plasma ions or dissolved oxygen.

The contrast agent composition of the invention may be in a ready to use concentration or may be a concentrate form for dilution prior to administration.

The contrast media compositions containing compounds of formula (I) and formula (II) can be administered by injection or infusion, e.g. by intervascular administration. Alternatively, contrast media compositions may also be administered orally. For oral administration the contrast medium may be in the form of a capsule, tablet or as liquid solution.

Hence, the invention further embraces use of a diagnostic composition containing compounds of formula (I) and formula (II) in X-ray contrast examinations and use of a compound of formula (I) and formula (II) for the manufacture of a diagnostic composition for use as an X-ray contrast agent.

A method of diagnosis comprising administration of compositions of formula (I) and formula (II) to the human or animal body, examining the body with a diagnostic device and compiling data from the examination is also provided. In the method of diagnosis the body may also be preadministrated with the composition.

Furthermore, a method of imaging, specifically X-ray imaging is provided, which comprises administration of compositions of formula (I) and formula (II) to the human or animal body, examining the body with a diagnostic device and compiling data from the examination and optionally analysing the data. In the method of imaging the body may also be preadministrated with compounds of formula (I).

The compounds of the general formula (I) can be synthesized by multistep procedures from starting materials that are either known from the state of art or that are commercially available or can readily be produced from commercially available materials. The known synthesis for the production of iodixanol can generally be adapted to produce compounds of formula (I).

### Preparation

The compounds of formula (I) can be prepared utilizing the general procedures for the preparation of triiodinated monomeric non-ionic compounds known from the literature, e.g. following the synthesis provided in U.S. Pat. Nos. 4,352,788, 4,364,921, 4,001,323, 4,341,756, 4,250,113, 5,035,877, 5,043,152, and patent application WO97/00240.

The compounds of the formulas (II) and (III) can be prepared following the general procedure:

Compounds of formula (IVa) and if necessary of formula (IVb)

R—NH(CHO) Formula (IVa)

R—NH(R³) Formula (IVb)

are reacted with a reactive linker group of formula (V)

Y-X-Y′ Formula (V)

wherein Y and Y′ are readily eliminatable atoms or groups and X has the above meaning or a hydroxyl protected derivative thereof or a corresponding epoxide in which one or both of the substituents Y and Y′ are replaced by —O—, and if required followed by removal of protecting groups. The groups Y and Y′ may be selected from the group of halogen atoms, e.g. chloride, bromine or iodine, or sulphate hydrocarbylsulphonyloxy groups, e.g. alkyl- or aryl-sulphonyloxy groups such as tosyloxy or mesyloxy.

Examples of suitable compounds of formula (V) are compounds of formulas (Va),(Vb), (Vc) and (Vd).

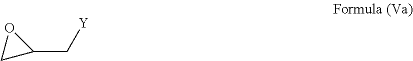

wherein Y is a readily eliminatable atom or group.

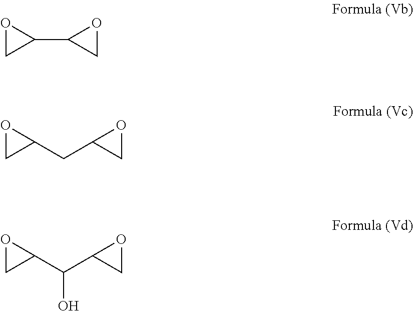

Further, compounds of formula (V) providing a bridge with 3 carbon atoms are described in Bjørsvik, H-R., and Priebe, H. Acta Chem. Scand. 49 (1995) 446-456, “Multivariate data analysis of molecular descriptors estimated by using semi-empirical quantum chemistry methods. Principal properties for synthetic screening of 2-chloromethyl-oxirane and analogues bis-alkylating C3 moieties”.

Suitable compounds of formula (V) may thus be epichlorohydrin, butadiene diepoxide, 1,4-pentadiene diepoxide, di(oxiran-2-yl)methanol or any precursor that can form epoxide or diepoxide under basic conditions like 1,4-dichloro-butane-2,3-diol or 1,5-dichloropentane-2,4-diol.

The hydroxyl groups present in the R groups and in the X group may, if desired, be in a hydroxyl protected form. Suitable protecting groups include acyl groups such as acetyl or, where adjacent hydroxyl groups are present, as cyclic ketal or acetal groups.

The reaction between compounds of formulas (IVa) and (V) and optionally between formulas (IVa), (IVb) and (V) is preferably effected in the presence of an acid binding agent, for example an organic or inorganic base preferably in aqueous or alcoholic medium or mixtures thereof such as water and/or an alkanol or glycol; an alkali metal alkoxide such as sodium metoxide or an alkali metal hydroxide such as sodium and potassium hydroxide may be used as base.

Any protecting group may be removed by standard methods, for example by hydrolysis. The compounds of formula (IVa) and (IVb) may be prepared by formylation of the corresponding compounds having free amino groups. In this reaction, hydroxyl groups in the substituents R may also be protected by acylation.

The compounds of formula (I) may be purified in any convenient manner, e.g. by preparative chromatography or by recrystallisation.

### Preparation of Intermediates (When Not Commercially Available)

The precursors to the compounds of formulas (IVa) and (IVb), the tri-iodinated phenyl groups having a free amino group are commercially available or can be produced following procedures described or referred to e.g. in WO95/35122 and WO98/52911. 5-amino-2,4,6-triiodo-isophtalic acid for example is available e.g. from Aldrich and 5-amino-2,4,6-triiodo-N,N′-bis(2,3-dihydroxypropyl)-isophtalamide is commercially available e.g. from Fuji Chemical Industries, Ltd.

Examples of commercial available precursors of the compounds of formulas (IVa) and (IVb), either commercially available or previously described in the literature include:

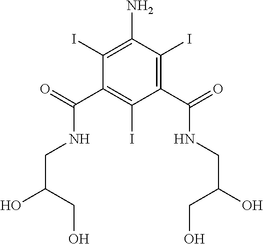

5-Amino-N,N′-bis-(2,3-dihydroxy-propyl)-2,4,6-triiodo-isophthalamide

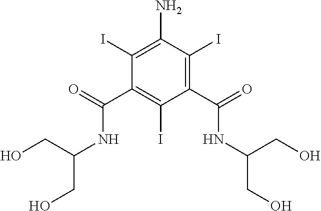

5-Amino-N-(2,3-dihydroxy-propyl)-N′-(2-hydroxy-1-hydroxymethyl-ethyl)-2,4,6-triiodo-isophthalamide (WO2002044125)

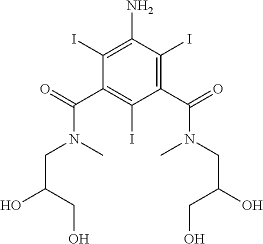

5-Amino-N,N′-bis-(2,3-dihydroxy-propyl)-2,4,6-triiodo-N,N′-dimethyl-isophthalamide

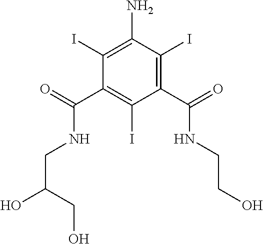

5-Amino-N-(2,3-dihydroxy-propyl)-N′-(2-hydroxy-ethyl)-2,4,6-triiodo-is ophthalamide (WO 8700757)

The compounds of formulas (IVa) and (IVb), may be prepared by acylation of the corresponding compounds having free amino groups. In this reaction, hydroxyl groups in the substituents R may also be protected by acylation.

Acylation may be effected by any convenient method, e.g. by use of activated formic acid such as mixed anhydrides which can prepared by a variety of methods described in the literature.

A convenient method of preparing mixed anhydrides is to add a carboxylic acid anhydride to an excess of formic acid under controlled temperature. It is also possible to make mixed anhydrides by addition of a carboxylic acid chloride to a solution of a formic acid salt. Formyl-mixed anhydrides may include acetyl, isobutyryl, pivaloyl, benzoyl etc.

In the present implementation acetic-formic mixed anhydride is employed. To an excess of cooled pre-prepared acetic-formic mixed anhydride is added a 5-amino-monomer and the mixture is stirred overnight. The mixture is concentrated in vacuo and may be used directly in the alkylation step as described in the experimental section (procedure B) or alternatively the O-acylated groups may be hydrolysed prior to alkylation as described in the experimental section (procedure A). Hydrolysis is conveniently performed in aqueous basic media as exemplified in the experimental section or may alternatively be effected by alcoholysis e.g. as described in WO1997000240.

It is also possible to dissolve the 5-aminomonomer in formic acid and subsequently add the carboxylic acid anhydride but in order to reduce unwanted acylation it is preferred to prepare the mixed anhydride separately and subsequently mix this with the 5-aminomonomer as described above.

More particularly, the compounds of formula IIIa above are prepared by following one of the procedures 1 or 2 below:

### Procedure 1

### 5,5′-(2-hydroxvpropane-1,3-diyl)bis(formylazanedivyl)bis(N¹,N³-bis(2,3-dihydroxvpropyl)-2,4,6-triiodoisophthalamide)

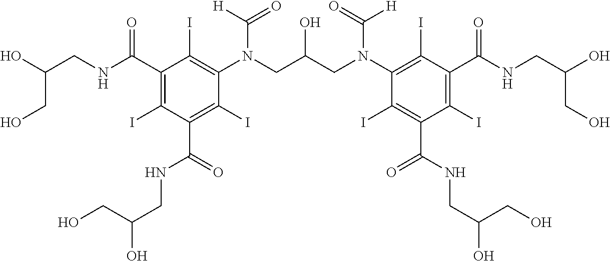

### 1a) N,N′-Bis-(2,3-dihydroxy-propyl)-5-formylamino-2,4,6-triiodo-isophthalamide

Formic acid (300 ml) was charged in a dry 1000 ml flask fitted with a dropping funnel, stir bar, thermometer and a gas inlet. The acid was cooled on an ice bath under a nitrogen blanket and acetic anhydride (144.8 g, 1.418 mol) was added drop wise at a rate so that the temperature did not exceed 2.5 C. After complete addition, the ice bath was removed and the temperature was allowed to reach 10° C. The mixture was again ice cooled and 5-amino-N,N′-bis(2,3-dihydroxypropyl)-2,4,6-triiodo-isophthalamide (100 g, 141.8 mmol) was added over 5 minutes and the mixture was left stirring over night while attaining ambient temperature. The mixture was evaporated to dryness and methanol (300 ml) and water (300 ml) was added. 2 M potassium hydroxide was added until all material was in solution and a stable pH 12.5 was attained. The methanol was removed in vacuo. The mixture was neutralized with 4 M HCl and a slow precipitation started. 300 ml water was added and the product was precipitated over night.

The precipitate was collected and rinsed with a small amount of water and dried on filter to a moist cake and further dried in vacuo to yield 84.8 g (81.5%) of N,N′-bis-(2,3-dihydroxy-propyl)-5-formylamino-2,4,6-triiodo-isophthalamide.

¹H-NMR 500 MHz (solvent: D₂O, ref. H₂O=4.8 ppm, 25° C.): 8.35 and 8.05 ppm (2s, 1H), 3.94 ppm (m, 2H), 3.67 ppm (m, 2H), 3.55 ppm (m, 2H), 3.45 ppm (m, 2H), 3.34 ppm (m, 2H).

LC-MS (column Agilent Zorbax SB-Aq 3.5 μm 3.0×100 mm, solvents: A=water/0.1% formic acid and B=acetonitrile/0.1% formic acid; gradient 0-30% B over 20 min; flow 0.3 ml/ min, UV detection at 214 and 254 nm, ESI-MS) gave two peaks centred at 5.5 minutes with m/z (M+H+) 733.828, m/z (M+NH₄+) 750.855, m/z (M+Na+) 755.817 corresponding to the structure.

### 1b) 5,5′-(2-hydroxypropane-1,3-diyl)bis(formylazanediyl)bis(N¹,N³-bis(2,3-dihydroxypropyl)-2,4,6-triiodoisophthalamide)

Potassium hydroxide (1.07 g) was dissolved in water (6.9 ml) and methanol (3.4 ml) in a 50 ml round bottomed flask fitted with a magnetic stir bar. Boric acid (0.41 g, 6.6 mmol) and N,N′-bis-(2,3-dihydroxy-propyl)-5-formylamino-2,4,6-triiodo-isophthalamide (7.0 g, 9.56 mmol) was added to the stirred solution.

Epichlorohydrin (260 ul, 3.32 mmol) was added to the solution and a pH electrode was fitted in the flask and the pH was maintained at pH 12.7 by drop wise addition of 4 M potassium hydroxide for 4 h. At this point, the mixture was left stirring over night. The pH was adjusted with 4 M hydrochloric acid to pH 4 and the methanol was removed in vacuo. The remaining aqueous solution was diluted with water (75 ml) and treated with ion exchangers (AMB200C and IRA67) to zero conductivity. The ion exchangers were removed by filtration and rinsed with water and the combined aqueous filtrates were freeze dried. The crude product was purified by preparative HPLC (column Phenomenex Luna C18 10 pm solvents: A=water and B=acetonitrile; gradient 05-20% B over 60 min. After freeze drying 3.80 g of 5,5′-(2-hydroxypropane-1,3-diyl)bis(formylazanediyl)bis(N¹,N³-bis(2,3-dihydroxypropyl)-2,4,6-triiodoisophthalamide) (74.8% yield) was obtained.

¹H-NMR 500 MHz (solvent: D₂O, ref. H₂O=4.8 ppm, 25° C.): 8.34 and 8.08 ppm (m, 2H), 2.80-4.80 ppm (m 26H).

LC-MS TOF; 1522.68 m/z (M+H⁺), 1544.66 m/z (M+Na⁺).

### Procedure 2

### 5,5′-(2-hydroxybrobane-1,3-diyl)bis(formylazanediyl)bis(N¹,N³-bis(2,3-dihydroxybrobyl)-2,4,6-triiodoisophthalamide)

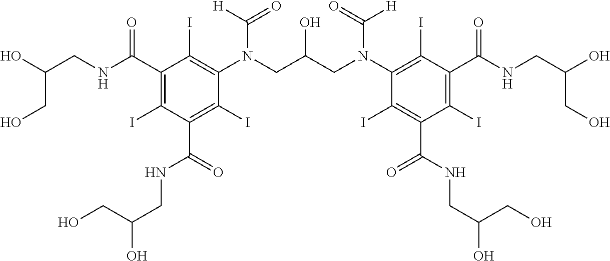

### 2a)1-formylamino-3,5-bis(2,3-bis(formyloxy)broban-1-ylcarbamoyl)-2,4,6-triiodo-benzene

Formic acid (4 L) was charged in a dry 5000 ml jacketed reactor on cryostat was fitted with a dropping funnel, mechanical stirring, thermometer and a gas inlet. The acid was cooled with a cryostat under a nitrogen blanket. Acetic anhydride (1.98 L, 21.0 mol) was added drop wise at a rate so that the temperature did not exceed 12.0° C. After 7.5 h the addition was completed and the mixture was cooled to 3.8° C. and 5-amino-N,N′-bis(2,3-dihydroxypropyl)-2,4,6-triiodo-isophthalamide (1.481 kg, 2.1 mol) was added over 20 minutes and the mixture was left stirring over night attaining ambient temperature.

The reaction mixture was evaporated in vacuo at 40° C. to a moist mass, this was further dried in a vacuum oven at 40° C. to yield 1754 g (98.8%) of 1-formylamino-3,5-bis(2,3-bis(formyloxy)propan-1-ylcarbamoyl)-2,4,6-triiodo-benzene. The product was used in the next step without purification.

The obtained product does contain some minor fraction of O-acetyl esters, as the product is used directly in the next step without purification this can be disregarded.

### 2b) 5,5′-(2-hydroxvpropane-1,3-diyl)bis(formylazanediyl)bis(N¹,N³-bis(2,3-dihydroxypropyl)-2,4,6-triiodoisophthalamide)

A 1000 ml jacketed reactor on cryostat was fitted with internal pH electrode, thermometer and stirrer. The reactor was cooled to 10° C., water (77 ml), methanol (154 ml) and boric acid (49.7 g, 803.5 mmol) were charged in the reactor. A slow addition of potassium hydroxide (9 M) was started and at T=0 finely crushed 1-formylamino-3,5-bis(2,3-bis(formyloxy)propan-1-ylcarbamoyI)-2,4,6-triiodo-benzene (341.5 g, 401.8 mmol) was added to the reactor. The addition rate of potassium hydroxide was adjusted to keep the pH within pH 11.6-11.7 and the temperature was maintained at 10° C. ±1. At T=105 minutes the starting material was largely in solution and epichlorohydrin (16.07 ml, 204.9 mmol) was added in 5 portions over 60 minutes. The pH was maintained within pH 11.6-11.7 by continuous addition of potassium hydroxide (9 M).

At T=465 minutes the pH was 11.7 and the mixture was left stirring over night at 10° C. without pH-adjustment. The following day the pH was maintained within pH 11.6-11.7 with continuous addition of potassium hydroxide (9 M). At the end of the day a temperature gradient of 1° C./h to 20° C. was started and the mixture was left stirring over the night. The following day the reaction mixture was diluted with water (500 ml) and taken out of the reactor and treated with acidic ion exchanger AMB200C (1841 ml, 3093.6 mmol). The pH was now pH 1.38. After 5 minutes basic ion exchanger IRA67 (2946 ml, 3093.6 mmol) was added and the pH gradually attained pH 5.67.After 4 h the ion exchangers were removed by filtration and rinsed with water (4×2 liters).

HPLC analysis (UV 254 nm) showed the product to be present in a purity of 90.4%.

The combined aqueous filtrates were combined and reduced to 1.5 liters in vacuo at 40° C.

The crude product was purified by preparative HPLC (column Phenomenex Luna C18 (2) 10 μm solvents: A=water and B=acetonitrile; gradient 05-20% B over 60 min. After freeze drying 222.8 g 5,5′-(2-hydroxypropane-1,3-diyl)bis(formylazanediyl)bis(N¹,N³-bis(2,3-dihydroxypropyl)-2,4,6-triiodoisophthalamide) (72.9% yield) was obtained.

LC-MS TOF 1522.68 m/z (M+H⁺), 1544.66 m/z (M+Na⁺).

¹H-NMR 500 MHz (solvent: D₂O, ref. H₂O=4.8 ppm, 25° C.): 8.34 and 8.08 ppm (m, 2H), 2.80-4.80 ppm (m 25H).

### BRIEF DESCRIPTION OF THE DRAWINGS

FIG. 1 depicts the viscosity of various mixtures of compound IIIa and iopamidol containing 350 mgl/ml.

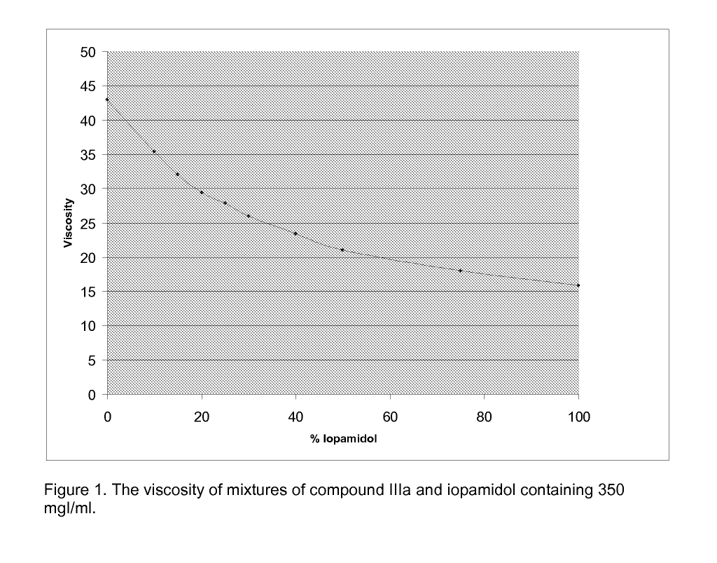

### EXAMPLES

### Example 1

### Preparation of a composition of 5,5′-(2-hydroxvpropane-1,3-diyl)bis(formylazanediyl)bis(N¹,N³-bis(2,3-dihydroxypropyl)-2,4,6-triiodoisophthalamide) (IIIa) and lopamidol

To a 50 ml flask was added Iopamidol (7.167 g) corresponding to 3.511 g iodine. The mass was corrected for 1.7% water content.

5,5′-(2-hydroxypropane-1,3-diyl)bis(formylazanediyl)bis(N¹,N³-bis(2,3-dihydroxypropyl)-2,4,6-triiodoisophthalamide) (28.579 g) corresponding to 14.295 g iodine (mass corrected for 3.0% water content) was added to the flask in two portions. Water was added in portions to the flask and the content was dissolved by occasional agitation and heating. When a clear solution was achieved water was added (a total of 32.851 g) to make 50 ml volume.

The clear solution was filtered through a Millipore Sterivex 0.22 um filter.

The obtained iodine concentration was 356 mgl/ml. The viscosity was determined to 28.7 mPas at 20° C. The osmolality was determined to 295 mOsm/kg.

### Example 2

### Compositions of Compound IIIa and Lopamidol in Different Proportions and Their Viscosities

a) Preparation of a 350 mgl/ml solution of IIIa containing 10 mM Tris and EDTA (0.1 mg/ml)

To a calibrated flask with a volume of 99.655 ml was added 71.174 g IIIa (water content 2.03%), 153 mg Trizma preset crystals (pH 7.3) and 10 mg EDTA disodium dihydrate followed by 67.330 g Milli-Q water. The mixture was brought to clear solution by intermittent agitation, gentle heating and sonication.

The clear solution with pH=6.95 was filtered through a Millipore Sterivex 0.22 um filter.

b) Preparation of a 350 mgl/ml solution of iopamidol containing 10 mM Tris and EDTA (0.1 mg/ml)

To a calibrated flask with a volume of 99.655 ml was added 72.429 g iopamidol (water content 1.7%), 157 mg Trizma preset crystals (pH 7.3) and 10 mg EDTA disodium dihydrate followed by 66.309 g Milli-Q water. The mixture was brought to clear solution by intermittent agitation, gentle heating and sonication.

The clear solution with pH=6.85 was filtered through a Millipore Sterivex 0.22 um filter.

c) The two solutions from a) and b) were mixed in different proportions according to Table 1. After thorough mixing, the viscosity was determined at 20° C. using conventional U-tube time of fall technique.

TABLE 1
The viscosity of mixtures of IIIa and iopamidol.

| % of IIIa in mixture | % of Iopamidol in mixture | Viscosity at 20° C. |
|---|---|---|
| (by volume) | (by volume) | (mPas) |
| 100 | 0 | 42.9 |
| 90 | 10 | 35.4 |
| 85 | 15 | 32.1 |
| 80 | 20 | 29.4 |
| 75 | 25 | 27.8 |
| 70 | 30 | 26.0 |
| 60 | 40 | 23.4 |
| 50 | 50 | 21.0 |
| 25 | 75 | 18.0 |
| 0 | 100 | 15.8 |

What is claimed is:

1. Contrast media compositions comprising iodine containing contrast enhancing compounds wherein
one or more compounds are of formula (I) 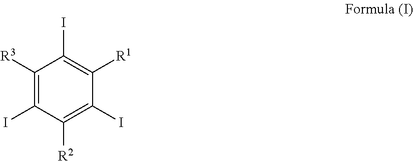
and salts or optical active isomers thereof,
wherein each of R¹, R² and R³ are the same or different and denotes a hydrogen atom or a non-ionic hydrophilic moiety, provided that at least one of the R¹, R² and R³ groups in the compound of formula (I) is a hydrophilic moiety,
and;
one or more compounds are of formula (II)
Formula (II)
R—N(CHO)—X—N(R⁶)—R (II)
and salts or optical active isomers thereof,
wherein
X denotes a C₃ to C₈ straight or branched alkylene moiety optionally with one or two CH₂ moieties replaced by oxygen atoms, sulphur atoms or NR⁴ groups and wherein the alkylene moiety optionally is substituted by up to six —OR⁴ groups;
R⁴ denotes a hydrogen or a C₁ to C₄ straight or branched alkyl group;
R⁶ denotes a hydrogen atom or an acyl function; and
each R independently is the same or different and denotes a triiodinated phenyl group further substituted by two groups R⁵ wherein each R⁵ is the same or different and denotes a hydrogen atom or a non-ionic hydrophilic moiety, provided that at least one R⁵ group in the compound of formula (II) is a hydrophilic moiety.

2. Compositions as claimed in claim 1 wherein in formula (I) the non-ionic hydrophilic moieties R¹, R² and R³ are the same or different and denote a non-ionic hydrophilic moiety comprising esters, amides or amine moieties, optionally further substituted by a straight chain or branched chain C₁₋₁₀ alkyl groups, where the alkyl groups may have one or more CH₂ or CH moieties replaced by oxygen or nitrogen atoms and may optionally contain one or more groups selected from oxo, hydroxyl, amino or carboxyl derivative, and oxo substituted sulphur and phosphorus atoms, and wherein each of the straight or branched alkyl groups optionally contains 1 to 6 hydroxy groups.

3. Compositions as claimed in claim 2 wherein the R¹, R² and R³ groups are the same or different and are polyhydroxy C₁₋₅ alkyl, hydroxyalkoxyalkyl with 1 to 5 carbon atoms or hydroxypolyalkoxyalkyl with 1 to 5 carbon atoms, and are attached to the iodinated phenyl group of formula (I) via amide or carbamoyl linkages.

4. Compositions as claimed in claim 3 wherein the R¹, R² and R³ groups of formula (I) are selected from the formulas
—CONH₂;
—CONHCH₃;
—CONH—CH₂—CH₂—OH₂;
—CONH—CH₂—CH₂—OCH₃;
—CONH—CH₂—CHOH-CH₂—OH;
—CONH—CH₂—CHOCH₃—CH₂—OH;
—CONH—CH₂—CHOH—CH₂—OCH₃;
—CON(CH₃)CH₂—CHOH-CH₂OH;
—CONH—CH—(CH₂—OH)₂;
—CON—(CH₂—CH₂—OH)₂;
—CON—(CH₂—CHOH—CH₂—OH)₂;
—CONH—OCH₃;
—CON (CH₂—CHOH—CH₂—OH)(CH₂—CH₂—OH)₂;
—CONH—C(CH₂—OH)₂CH₃;
—CONH—C(CH₂—OH)₃;
—CONH—CH(CH₂—OH)(CHOH—CH₂—OH);
—NH(COCH₃);
—N(COCH₃)C₁₋₃ alkyl;
—N(COCH₃)-mono, bis or tris-hydroxy C₁₋₄ alkyl;
—N(COCH₂OH)-hydrogen, C₁₋₄ alkyl, mono, bis or tris-hydroxy C₁₋₄ alkyl;
—N(CO—CHOH—CH₂OH)-hydrogen, mono, bis or trihydroxylated C₁₋₄ alkyl;
—N(CO—CHOH—CHOH—CH₂OH)-hydrogen, mono, bis or trihydroxylated C₁₋₄ alkyl;
—N(CO—CH—(CH₂OH)₂)-hydrogen, mono, bis or trihydroxylated C₁₋₄ alkyl;
—N(CO—CHOH—CH₃)-hydrogen, mono, bis or trihydroxylated C₁₋₄ alkyl;
—NH(CO—CH₂OCH₃); and
—N(COCH₂OH)₂.

5. (canceled)

6. Compositions as claimed in claim 1 wherein the compounds of formula (I) are selected from the compounds iopamidol, iomeprol, ioversol, iopromide, iobitridol, iopentol and iohexol.

7. Compositions as claimed in claim 6 wherein the compounds of formula (I) are selected from the compounds iopamidol and iohexol.

8. Compositions as claimed in claim 1 wherein for the compounds of formula (II) X denotes a straight C₃ to C₈ alkylene chain optionally substituted by one to six —OR⁴ groups.

9. Compositions as claimed in claim 8 wherein the R⁴ group of formula (II) denotes a hydrogen atom or a methyl group.

10. Compositions as claimed in claim 1 wherein X denotes a straight C₃ to C₅ alkylene chain having at least one hydroxyl group substituted in a position that is not vicinal to the bridge nitrogen atom.

11. (canceled)

12. Compositions as claimed in claim 10 wherein X comprises 2-hydroxy propylene, 2,3-dihydroxy butylene, 2,4-dihydroxy pentylene and 2,3,4-trihydroxy pentylene linkers.

13. Compositions as claimed in claim 1 wherein the R⁶ group of formula (II) denotes a hydrogen atom or a residue of an aliphatic organic acid selected from formyl, acetyl, propionyl, butyryl, isobutyryl and valeriyl moieties.

14. (canceled)

15. Compositions as claimed in claim 1 wherein each of the triiodinated phenyl group R of formula (II) denotes a 2,4,6-triiodinated phenyl group further substituted by two groups R⁵ in the remaining 3 and 5 positions in the phenyl moiety, wherein each R⁵ are the same or different and denotes a non-ionic hydrophilic moiety comprising esters, amides or amine moieties, optionally further substituted by a straight chain or branched chain C₁₋₁₀ alkyl groups, optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atom, and optionally further substituted by a straight chain or branched chain C₁₋₅ alkyl groups substituted by 1 to 3 hydroxy groups.

16. (canceled)

17. Compositions as claimed in claim 15 wherein each R⁵ is the same or different and is selected from groups of the formulas
—CONH₂;
—CONHCH₃;
—CONH—CH₂—CH₂—OH;
—CONH—CH₂—CH₂—OCH₃;
—CONH—CH₂—CHOH—CH₂—OH;
—CONH—CH₂—CHOCH₃—CH₂—OH;
—CONH—CH₂—CHOH—CH₂—OCH₃;
—CON(CH₃)CH₂—CHOH—CH₂OH;
—CONH—CH—(CH₂—OH)₂;
—CON—(CH₂—CH₂—OH)₂;
—CON—(CH₂—CHOH—CH₂—OH)₂;
—CONH—OCH₃;
—CON(CH₂—CHOH—CH₂—OH)(CH₂—CH₂—OH);
—CONH—C(CH₂—OH)₂CH₃;
—CONH—C(CH₂—O H)₃;
—CONH—CH(CH₂—OH)(CHOH—CH₂—OH);
—NH(COCH₃);
—N(COCH₃)C₁₋₃ alkyl;
—N(COCH₃)-mono, bis or tris-hydroxy C₁₋₄ alkyl;
—N(COCH₂OH)-hydrogen, mono, bis or tris-hydroxy C₁₋₄ alkyl;
—N(CO—CHOH-CH₂OH)-hydrogen, mono, bis or trihydroxylated C₁₋₄ alkyl;
—N(CO—CHOH—CHOH—CH₂OH)-hydrogen, mono, bis or trihydroxylated C₁₋₄ alkyl;
—N(CO—CH—(CH₂OH)₂)-hydrogen, mono, bis or trihydroxylated C₁₋₄ alkyl; and
—N(COCH₂OH)₂.

18. (canceled)

19. Compositions as claimed in claim 1 wherein the compounds of formula (II) are selected from formulas (IIa), (IIb) and (IIc)
R—N(CHO)—X—N(CHO)—R (IIa)
R—N(CHO)—X—N(CO(CH₃))—R (IIb)
R—N(CHO)—X—NH—R (IIc)
wherein R and X are as defined in claim 1.

20. Compositions as claimed in claim 1 wherein the compounds of formula II are selected from the group of:
5,5′-(2-hydroxypropane-1,3-diyl)bis(formylazanediyl)bis(N¹,N³-bis(2,3-dihydroxypropyl)-2,4,6-triiodoisophthalamide);
5,5′-(2,3-dihydroxybutane-1,4-diyl)bis(formylazanediyl)bis(N¹,N³-bis(2,3-dihydroxypropyl)-2,4,6-triiodoisophthalamide);
5,5′-(2,4-dihydroxypentane-1,5-diyl)bis(formylazanediyl)bis(N¹,N³-bis(2,3-dihydroxypropyl)-2,4,6-triiodoisophthalamide);
5,5′-(2,3,4-trihydroxypentane-1,5-diyl)bis(formylazanediyl)bis(N¹,N³-bis(2,3-dihydroxypropyl)-2,4,6-triiodoisophthalamide);
5,5′-(2,3-dihydroxybutane-1,4-diyl)bis(formylazanediyl)bis(N¹,N³-bis(2,3-dihydroxypropyl)-2,4,6-triiodo-N¹,N³-dimethylisophthalamide);
5,5′-(2,4-dihydroxypentane-1,5-diyl)bis(formylazanediyl)bis(N¹,N³-bis(2,3-dihydroxypropyl)-2,4,6-triiodo-N¹,N³-dimethylisophthalamide);
5,5′-(2-hydroxypropane-1,3-diyl)bis(formylazanediyl)bis(N¹,N³-bis(2,3-dihydroxypropyl)-2,4,6-triiodo-N¹,N³-dimethylisophthalamide);
5,5′-(2-hydroxypropane-1,3-diyl)bis(formylazanediyl)bis(N¹,N³-bis(1,3-dihydroxypropan-2-yl)-2,4,6-triiodoisophthalamide);
5,5′-(2,3-dihydroxybutane-1,4-diyl)bis(formylazanediyl)bis(N¹,N³-bis(1,3-dihydroxypropan-2-yl)-2,4,6-triiodoisophthalamide);
5,5′-(2,4-dihydroxypentane-1,5-diyl)bis(formylazanediyl)bis(N¹,N³-bis(1,3-dihydroxypropan-2-yl)-2,4,6-triiodoisophthalamide);
5,5′-(2,3-dihydroxybutane-1,4-diyl)bis(formylazanediyl)bis(N¹-(2,3-dihydroxypropyl)-N³-(2-hydroxyethyl)-2,4,6-triiodoisophthalamide);
5,5′-(2-hydroxypropane-1,3-diyl)bis(formylazanediyl)bis(N¹-(2,3-dihydroxypropyl)-N³-(2-hydroxyethyl)-2,4,6-triiodoisophthalamide);
5,5′-(2,4-dihydroxypentane-1,5-diyl)bis(formylazanediyl)bis(N¹-(2,3-dihydroxypropyl)-N³-(2-hydroxyethyl)-2,4,6-triiodoisophthalamide);
5,5′-(2-hydroxypropane-1,3-diyl)bis(formylazanediyl)bis(N¹-(1,3-dihydroxypropan-2-yl)-N³-(2,3-dihydroxypropyl)-2,4,6-triiodoisophthalamide);
5,5′-(2,3-dihydroxybutane-1,4-diyl)bis(formylazanediyl)bis(N¹-(1,3-dihydroxypropan-2-yl)-N³-(2,3-dihydroxypropyl)-2,4,6-triiodoisophthalamide);
5,5′-(2,4-dihydroxypentane-1,5-diyl)bis(formylazanediyl)bis(N¹-(1,3-dihydroxypropan-2-yl)-N³-(2,3-dihydroxypropyl)-2,4,6-triiodoisophthalamide);
5,5′-(2-hydroxypropane-1,3-diyl)bis(formylazanediyl)bis(N¹-(1,3-dihydroxypropan-2-yl)-N³-(2-hydroxyethyl)-2,4,6-triiodoisophthalamide);
5,5′-(2,3-dihydroxybutane-1,4-diyl)bis(formylazanediyl)bis(N¹-(1,3-dihydroxypropan-2-yl)-N³-(2-hydroxyethyl)-2,4,6-triiodoisophthalamide);
5,5′-(2,4-dihydroxypentane-1,5-diyl)bis(formylazanediyl)bis(N¹-(1,3-dihydroxypropan-2-yl)-N³-(2-hydroxyethyl)-2,4,6-triiodoisophthalamide);
5-(N-(3-(N-(3,5-bis(2,3-dihydroxypropylcarbamoyl)-2,4,6-triiodophenyl)acetamido)-2-hydroxypropyl)formamido)-N¹,N³-bis(2,3-dihydroxypropyl)-2,4,6-triiodoisophthalamide; and
5-(3-(N-(3,5-bis(2,3-dihydroxypropylcarbamoyl)-2,4,6-triiodophenyl)formamido)-2-hydroxypropylamino)-N¹,N³-bis(2,3-dihydroxypropyl)-2,4,6-triiodoisophthalamide.

21. Compositions as claimed in claim 20 comprising at least one of iopamidol or iohexol and at least one of the compounds of claim 20.

22. Compositions as claimed in claim 1 comprising iopamidol or iohexol and 5,5′-(2-hydroxypropane-1,3 diyl)bis(formylazanediyl)bis(N¹,N³-bis(2,3-dihydroxypropyl)-2,4,6-triiodoisophthalamide).

23-26. (canceled)

27. Compositions as claimed in claim 2 wherein in formula (I) said non-ionic hydrophilic moieties R¹, R² and R³ are optionally further substituted by a straight chain or branched chain C₁₋₅ alkyl groups, where the alkyl groups may have one or more CH₂ or CH moieties replaced by oxygen or nitrogen atoms and may optionally contain one or more groups selected from oxo, hydroxyl, amino or carboxyl derivative, and oxo substituted sulphur and phosphorus atoms, and wherein each of the straight or branched alkyl groups optionally contains 1 to 3 hydroxy groups.

28. Compositions as claimed in claim 13 wherein the R⁶ group of formula (II) denotes a hydrogen atom or a formyl moiety.
